SEQUENCE LISTING (18 sequences)

SEQ ID NO: 1 - DNA, 20 bp, Artificial
  CpG oligonucleotide
tccatgacgttcctgacgtt

SEQ ID NO: 2 - DNA, 6 bp, Artificial
  CpG oligonucleotide
gacgtt

SEQ ID NO: 3 - DNA, 8 bp, Artificial
  CpG oligonucleotide
tgacgttc

SEQ ID NO: 4 - DNA, 10 bp, Artificial
  CpG oligonucleotide
atgacgttcc

SEQ ID NO: 5 - DNA, 12 bp, Artificial
  CpG oligonucleotide
catgacgttcct

SEQ ID NO: 6 - DNA, 14 bp, Artificial
  CpG oligonucleotide
ccatgacgttcctg

SEQ ID NO: 7 - DNA, 16 bp, Artificial
  CpG oligonucleotide
tccatgacgttcctga

SEQ ID NO: 8 - DNA, 16 bp, Artificial
  CpG oligonucleotide
tcctcaacgttcctga

SEQ ID NO: 9 - DNA, 16 bp, Artificial
  CpG oligonucleotide
tccgcaacgttcctga

SEQ ID NO: 10 - DNA, 16 bp, Artificial
  CpG oligonucleotide
tcctcgacgtccctga

SEQ ID NO: 11 - DNA, 16 bp, Artificial
  CpG oligonucleotide
tcctcagcgctcctga

SEQ ID NO: 12 - DNA, 16 bp, Artificial
  CpG oligonucleotide
tcctcaacgctcctga

SEQ ID NO: 13 - DNA, 16 bp, Artificial
  CpG oligonucleotide
tcctcatcgatcctga

SEQ ID NO: 14 - DNA, 16 bp, Artificial
  CpG oligonucleotide
tcctcttcgaacctga

SEQ ID NO: 15 - DNA, 17 bp, Artificial
  CpG oligonucleotide
tccatgacgttcctgac

SEQ ID NO: 16 - DNA, 18 bp, Artificial
  CpG oligonucleotide
tccatgacgttcctgacg

SEQ ID NO: 17 - DNA, 19 bp, Artificial
  CpG oligonucleotide
tccatgacgttcctgacgt

SEQ ID NO: 18 - DNA, 10 bp, Artificial
  Consensus Sequence
agagcgctct

(19) United States
(12) Patent Application Publication
(10) Pub. No.: US 2005/0209183 A1
(43) Pub. Date: Sep. 22, 2005

(54) Cosmetic or pharmaceutical preparations comprising nucleic acids based on non-methylated CPG motifs

(75) Inventors: Stefan Kippenberger, Frankfurt am Main (DE); August Bernd, Bingen-Kempten (DE); Roland Kaufmann, Muehlheim (DE)

Correspondence Address: WOODCOCK WASHBURN LLP, ONE LIBERTY PLACE, 46TH FLOOR, PHILADELPHIA, PA, 19103, US

(73) Assignee: PHENION GMBH & CO. KG, Frankfurt (DE)

(21) Appl. No.: 11/040,373

(22) Filed: Jan. 21, 2005

Related U.S. Application Data
(63) Continuation of application No. PCT/EP03/07748, filed on Jul. 17, 2003.

(30) Foreign Application Priority Data
Jul. 25, 2002 (DE) DE 102 33 994.5

(51) Int. Cl. A61K 48/00

(52) U.S. Cl. 514/44

(57) ABSTRACT

Embodiments of the present invention provide methods for the prophylaxis and/or treatment of epithelial integument with inflammatory changes that comprise administering to a patient suffering from such a disorder a pharmaceutically acceptable amount of a nucleic acid comprising a non-methylated CpG motif. Other aspects of the invention relate to cosmetic or pharmaceutical compositions that comprise nucleic acids comprising a non-methylated CpG motif, and to methods of preparing such compositions.

### CROSS-REFERENCE TO RELATED APPLICATIONS

This application is a continuation of PCT/EP2003/007748, filed Jul. 17, 2003, which claims priority to DE 102 33 994.5, filed Jul. 25, 2002, the disclosures of which are incorporated herein in their entireties.

### FIELD OF THE INVENTION

The present invention relates to cosmetic or pharmaceutical preparations for the prophylaxis and/or treatment of epithelial integument, which comprise nucleic acids based on non-methylated CpG motifs, to the use of such nucleic acids based on non-methylated CpG motifs for the prophylaxis and/or treatment of epithelial integument, and to softeners, handwashing compositions, body- and hair-care compositions, hair-coloring compositions or manual dishwashing compositions, comprising such nucleic acids based on non-methylated CpG motifs.

### BACKGROUND OF THE INVENTION

Unmethylated CG-rich sequences (CpG) are widespread in the bacterial genome, whereas they occur distinctly less commonly in the mammalian genome.

There has been evidence of immunostimulatory effects of “foreign” DNA since the 1960s (Jensen, K. E., Neal, A. L., Owens, R. E. and Warren, J. Interferon Responses of Chick Embryo Fibroblasts to Nucleic Acids and Related Compounds Nature 200 (1963) 433-434). There has been description both of the induction of the production of interferon gamma, and of the activation of natural killer cells, as well as induction of an antitumor activity by Bacille Calmette-Guerin (BCG) fractions (Tokunaga, T., Yamamoto, H., Shimada, S., Abe, H., Fukuda, T., Fujisawa, Y., Furutani, Y., Yano, O., Kataoka, T., Sudo, T., Makiguchi, N. and Suganuma, T. Antitumor Activity of Deoxyribonucleic Acid Fraction From Mycobacterium Bovis BCG I. Isolation, Physicochemical Characterization and Antitumor Activity J. Natl. Cancer Res. 72 (1984) 955-962). It was possible to destroy the immunostimulating activity of this fraction by preincubation with DNAses, but not with RNAses. This suggests that the bacterial DNA constitutes the immunostimulating portion of the BCG fraction. Detailed investigation of the immunologically active DNA sequences revealed that these are oligonucleotides consisting of a central palindrome with a CpG motif.

Further investigations gave rise to the theory that the motif important for the immunostimulatory effect is composed of a central CpG group flanked at the 5′ end by two purines and at the 3′ end by two pyrimidines (Krieg, A. M., Yi, A., Matson, S., Waldschmidt, T. J., Bishop, G. A., Teasdale, R., Koretzky, G. A. and Klinman, D. M. CpG Motifs in Bacterial DNA Trigger direct B-Cell Activation Nature 374 (1995) 546-549). CpG Dinucleotides are suppressed in eukaryotic DNA.

On the one hand, these motifs occur in eukaryotic DNA with only one fifth of the expected frequency and, on the other hand, they are 60-90% methylated (Bird, A. P. CpG-rich Islands and the Function of DNA Methylation Nature 321 (1986) 209-213). In contrast thereto, the CpG motif is found unmethylated in bacterial DNA and with the expected frequency (1:16). It has been possible to show that the methylation destroys the stimulatory potential of the CpG motif (Krieg, see above). These differences between bacterial and eukaryotic DNA make sensible interpretation possible of the biological observations concerning the immunostimulatory effect of bacterial DNA and synthetic CpG oligodeoxynucleotides (ODN).

The recognition of “foreign” DNA and the subsequent immunological response is one possibility for the innate immune system to distinguish between “self” and “foreign” without being dependent on the mediation and intervention of the adaptive immune system.

The effects and mechanism of action of CpG ODNs have been investigated in particular in the murine system. ODNs have stimulatory effects both on the innate and on the adaptive immune system of the mouse, the effects differing in relation to signal transmission and sequence specificity. It has been possible to show for example that CpG ODNs have immunostimulatory effects on the various types of antigen-presenting cells (APC). Stimulation of purified B lymphocytes with unmethylated CpG ODNs leads to proliferation and secretion of immunoglobulins (Krieg, see above). In macrophages, CpG ODNs induce activation of the transcription factor Nuclear Factor kB (NF-kB), transcription of cytokine mRNA and secretion of cytokines such as TNFα, IL-1, IL-6 and IL-12 (Sparwasser, T., Miethke, T. and Lipford, G. B. Macrophages Sense Pathogens via DNA Motifs: Induction of Tumor Necrosis Factor-Alpha-Mediated Shock Eur. J. Immunol. 27 (1997) 1671-1679).

CpG ODNs have activating effects both on mature and on immature dendritic cells. In both cell populations, they enhance MHC II expression and the expression of costimulatory molecules (CD40, CD86) and induce the production of cytokines such as IL-6, IL-12 and TNFα. In order to investigate the mechanism of action of CpG ODNs in detail, experiments have been carried out with immobilized CpG ODNs. The results of these experiments indicate that uptake of CpG ODNs into the cell is necessary for the immunostimulatory effect (Krieg, see above).

Other experiments show that uptake of the DNA on the cell surface of macrophages can be blocked by any competitively added ODN (Häcker, H., Mischak, H., Miethke, T., Liptary, S., Schmid, R., Sparwasser, T., Heeg, K., Lipford, G. B. and Wagner, H. CpG-DNA-Specific Activation of Antigen-Presenting Cells Requires Stress Kinase Activity and Is Preceded by Non-Specific Endocytosis and Endosomal Maturation EMBO Journal 17 (1998) 6230-6240), which suggests that uptake of the ODNs into the cell does not take place sequence-specifically. On the other hand, the CpG specificity might be mediated by an intracellular receptor located, for example, in the endosome.

The theory of the endosomal location of an intracellular CpG receptor is supported by results showing that endosomal acidification is necessary for the CpG ODN signal pathway, because the effect of CpG ODNs is blocked by inhibitors of endosomal acidification such as, for example, chloroquine (Häcker, see above).

The complete mechanism of action and signal pathway of CpG ODNs is, however, for the most part still unexplained. CpG ODNs exert their effect on macrophages and dendritic cells directly, whereas the effect of CpG ODNs on B cells is possible both directly and in the sense of costimulation (Krieg, see above). On the other hand, it has not been possible to show direct effects of CpG ODNs on T cells. However, T cells which receive their signal via a T-cell receptor ligation are costimulated by CpG ODNs (Bendigs, S., Salzer, U., Lipford, G. B., Wagner, H. and Heeg, K. CpG Oligodeoxynucleotides Co-Stimulate Primary T Cells in the Absence of Antigen-Presenting Cells Eur. J. Immunol. 29 (1999) 1209-1218).

These results suggest that the mechanism of T-cell costimulation differs from that of a direct effect of CpG ODNs in APC. The investigations of the direct effect of ODNs on T cells were carried out in vitro. However, there are many in vivo results in which CpG ODNs were used as adjuvants, and T-cell activation was induced by injection of CpG ODNs in vivo. The CpG ODNs assist the development of a TH1 immune response and induce a strong peptide-specific cytotoxic T-lymphocyte activity (CTL).

In contrast to the murine system, there are few findings on the effect of ODNs in the human system. It is known that ODNs with CpG motifs induce the production of IFNα in peripheral blood lymphocytes. It has also been shown that natural killer cells are isolated by CpG ODNs—similar to the murine system—through the mediation of IL-12 which is produced by activated macrophages (Ballas, Z. K., Rasmussen, W. L. and Krieg, A. M. Induction of NK Activity in Murine and Human Cells by CpG Motifs in Oligodeoxynucleotides and Bacterial DNA J. Immunol. 157 (1996) 1840-1845). Human peripheral mononuclear cells (peripheral blood mononuclear cells=PBMC) are sequence-specifically activated by CpG ODNs.

This activation leads to increased expression of CD86, CD40, MHC I and MHC II molecules and to a production of cytokine IL-12, IL-6 and TNFα. As in the murine system, CpG ODNs induce the proliferation of human B cells (Bauer, M., Heeg, K., Wagner, H. and Lipford G. B. DNA Activates Human Immune Cells through a CpG Sequence-Dependent Manner Immunology, 97 (1999) 699-705). The results obtained to date in the human system have much in common with the results in the murine system.

An immunoprotective and immunostimulating effect is therefore ascribed to CpGs in the art, and they are also employed with this in view—still experimentally at present. A suppressant effect of CpGs on the immune system, especially on the immune system of the skin, has not, on the other hand, been disclosed.

### SUMMARY OF THE INVENTION

It has surprisingly been found that CpGs exert an immunosuppressant effect on topical use on the skin. Specifically, the expression of proinflammatory cytokines and chemokines (IL-6 and IL-8) in skin cells is suppressed through the use of CpGs.

### BRIEF DESCRIPTION OF THE DRAWINGS

FIG. 1 depicts the results of experiments demonstrating that basal IL-8 expression is suppressed concentration-dependently by CpG. HaCaT cells were treated with 2, 4 and 6 μM CpG-1-PTO (5-tcc atg acg ttc ctg acg tt-3) (SEQ ID NO:1) for 18 h. The cell-free supernatants were then investigated for IL-8 in an ELISA.

FIG. 2 depicts the results of experiments demonstrating that basal IL-8 expression is suppressed concentration-dependently by chemically modified CpG. HaCaT cells were treated with 2, 4 and 6 μM CpG-1 (5-tcc atg acg ttc ctg acg tt-3) (SEQ ID NO:1) for 18 h. The cell-free supernatants were then investigated for IL-8 in an ELISA. Both phosphorothioates (PTO) and phosphodiester compounds (PDE) bring about suppression of IL-8 release.

FIG. 3 depicts the results of experiments demonstrating that TNFα-induced IL-8 release is suppressed by CpG. HaCaT cells were pretreated with 4 μM CpG-1-PTO (5-tcc atg acg ttc ctg acg tt-3) (SEQ ID NO:1) for 4 h. The cells were then stimulated in the presence of CpG-1-PTO with 20 ng/ml TNFα. After 18 h, the cell-free supernatants were investigated for IL-8 in an ELISA.

FIG. 4 depicts the results of experiments demonstrating that TNFα-induced IL-6 release is suppressed by CpG. HaCaT cells were pretreated with 4 μM CpG-1-PTO (5-tcc atg acg ttc ctg acg tt-3) (SEQ ID NO:1) for 4 h. The cells were then stimulated in the presence of CpG-1-PTO with 20 ng/ml TNFα. After 18 h, the cell-free supernatants were investigated for IL-6 in an ELISA.

FIG. 5 depicts the results of experiments demonstrating that anisosmotically induced IL-8 release is suppressed by CpG. HaCaT cells were pretreated with 4 μM CpG-1-PTO (5-tcc atg acg ttc ctg acg tt-3) (SEQ ID NO:1) for 4 h. The cells were then kept in the presence of CpG-1-PTO under hypoosmolar (−100 mM NaCl=−200 mosmol) conditions. After 18 h, the cell-free supernatants were investigated for IL-8 in an ELISA.

FIG. 6 depicts the results of experiments demonstrating that UVB light-induced IL-6 release is suppressed by CpG. HaCaT cells were pretreated with 4 μM CpG-1PTO (5-tcc atg acg ttc ctg acg tt-3) (SEQ ID NO:1) for 4 h. The cells were then stimulated in the presence of CpG-1-PTO with 150 mJ/cm2 UVB. After 18 h, the cell-free supernatants were investigated for IL-6 in an ELISA.

FIG. 7 depicts the immunosuppressant effect of CpG on skin.

FIG. 8 depicts the results of experiments demonstrating that basal IL-8 expression is suppressed by longer CpG (CpG 13-17). HaCaT cells were treated with the CpG-PTO shown in the stated concentrations for 18 h. The cell-free supernatants were then investigated for IL-8 in an ELISA.

FIG. 9 depicts the results of experiments in which the level of suppression of basal IL-8 expression resulting from various CpG is compared. HaCaT cells were treated with the CpG-PTO shown in the stated concentrations for 18 h. The cell-free supernatants were then investigated for IL-8 in an ELISA.

### DETAILED DESCRIPTION OF ILLUSTRATIVE EMBODIMENTS

Inflammations of the cutaneous system are widespread disorders which may be triggered both endogenously and exogenously. Treatment usually involves topical or systemic administration of steroid-based therapeutic agents. Besides its activity, this class of substances also shows a number of unwanted side effects (e.g. cutaneous atrophy, Cushing's syndrome). An alternative principle for the treatment of skin inflammations is therefore desirable.

The present invention therefore relates to a cosmetic or pharmaceutical preparation for the prophylaxis and/or treatment of epithelial integument, in particular for the prophylaxis and/or treatment of epithelial integument with inflammatory changes, which is characterized in that it comprises nucleic acids based on non-methylated CpG motifs.

Nucleic acids based on non-methylated CpG motifs mean nucleic acids which include at least one non-methylated central CG dinucleotide. The non-methylated CG dinucleotide is preferably flanked at the 5′ end by two purines (Pu) and on the 3′ side by two pyrimidines (Py). Nucleic acids based on non-methylated CpG motifs which can be employed according to the invention particularly preferably include at least one sequence which corresponds to the extent of 80% to 100%, preferably of 85% to 100%, in particular of 90% to 100%, particularly preferably of 95% to 100% and very particularly preferably of 100%, to the consensus sequence 5′-A/GA/GCGC/TC/T-3′ (SEQ ID NO:18), as described by Jakob et al., J. Imunology, 1998, 161:3042-49.

Nucleic acids based on non-methylated CpG motifs which are suitable according to the invention have a length of from 6 to 40, in particular 14 to 40, preferably 14 to 30, more preferably 14 to 25 and very particularly preferably from 14 to 20, nucleotides.

Particularly suitable nucleic acids are detailed for example in the sequence listing of WO 01/32877, which is incorporated herein by reference.

Very particularly suitable nucleic acids based on non-methylated CpG motifs are those including a sequence selected from

| | |
|---|---|
| CpG-1-PTO: | |
| 5′-TCC ATG ACG TTC CTG ACG TT-3′; | (SEQ ID NO: 1) |
| CpG-9-PTO: | |
| 5′-G ACG TT-3′; | (SEQ ID NO: 2) |
| CpG-10-PTO: | |
| 5′-TG ACG TTC-3′; | (SEQ ID NO: 3) |
| CpG-11-PTO: | |
| 5′-ATG ACG TTC C-3′; | (SEQ ID NO: 4) |
| CpG-12-PTO: | |
| 5′-C ATG ACG TTC CT-3′; | (SEQ ID NO: 5) |
| CpG-13-PTO: | |
| 5′-CC ATG ACG TTC CTG-3′; | (SEQ ID NO: 6) |
| CpG-14-PTO: | |
| 5′-TCC ATG ACG TTC CTG A-3′; | (SEQ ID NO: 7) |
| CpG-14A-PTO: | |
| 5′-TCC TCA ACG TTC CTG A-3′; | (SEQ ID NO: 8) |
| CpG-14B-PTO: | |
| 5′-TCC GCA ACG TTC CTG A-3′; | (SEQ ID NO: 9) |
| CpG-14C-PTO: | |
| 5′-TCC TCG ACG TCC CTG A-3′; | (SEQ ID NO: 10) |
| CpG-14D-PTO: | |
| 5′-TCC TCA GCG CTC CTG A-3′; | (SEQ ID NO: 11) |
| CpG-14E-PTO: | |
| 5′-TCC TCA ACG CTC CTG A-3′; | (SEQ ID NO: 12) |
| CpG-14F-PTO: | |
| 5′-TCC TCA TCG ATC CTG A-3′; | (SEQ ID NO: 13) |
| CpG-14G-PTO: | |
| 5′-TCC TCT TCG AAC CTG A-3′; | (SEQ ID NO: 14) |
| CpG-15-PTO: | |
| 5′-TCC ATG ACG TTC CTG AC-3′; | (SEQ ID NO: 15) |
| CpG-16-PTO: | |
| 5′-TCC ATG ACG TTC CTG ACG-3′; | (SEQ ID NO: 16) |
| and | |
| CpG-17-PTO: | |
| 5′-TCC ATG ACG TTC CTG ACG T-3′. | (SEQ ID NO: 17) |

The nucleic acids based on non-methylated CpG motifs which can be employed according to the invention may be completely (all nucleotides) or partially (only some nucleotides) chemically modified in a manner known to the skilled worker. Examples of preferred modifications are:
a) modification of the internucleoside bridges: replacement of phosphodiesters by methylphosphonates, phosphoramidates, phosphorothioates or hydroxylamines;
b) modification of the sugar components: replacement of ribose by various hexo- or pentopyranoses or 3′-5′-carbocyclically bridged derivatives of 2′-deoxyribose (Steffens R & Leumann C J (1997) Tricyclo-DNA: A phosphodiester-backbone based DNA analog exhibiting strong complementary base-pairing properties. J. Am. Chem. Soc. 119, 11548-11549);
c) replacement of strand backbone: replacement of the polyester chains based on sugar-phosphate units by carboxamide chains based on amino acid derivatives such as N-(2-aminoethyl)glycine units.

Hybrid molecules consisting of CpG-containing DNA/RNA sequences are particularly preferred according to the invention.

For the purposes of the present invention, epithelial integument means on the one hand the skin (consisting of subcutis, corium and epidermis) covering the outer surface of the body, and on the other hand the tissue lining the hollow organs and body cavities, including the epithelia of the uterus and of the mouth.

“With inflammatory changes” means for the purposes of the present invention “affected by an acute or chronic inflammation”. The inflammation may be caused by biological (e.g. pathogens, autoimmune reactions, TNF), chemical (e.g. poisons, irritants) or physical (e.g. UV radiation, osmotic changes, mechanical stress, thermal stress) noxae or stressors.

An acute inflammation is characterized by sudden occurrence with rapid, often severe progression over hours or days.

The chief symptoms of an acute inflammation are rubor (reddening due to vasodilatation), tumor (tissue swelling due to inflammatory exudate), calor (warming because of the increased blood flow through the tissue), dolor (pain due to nerve irritation) and function laesa (impaired function).

The various phases of an acute inflammation are controlled by the following mediators:
a) cellular mediators: biogenic vasoactive amines (histamine and serotonin), arachidonic acid derivatives (leukotrienes, prostaglandins, prostacyclin, thromboxane A2), platelet-activating factor (PAF), cytokines (interleukins, TNF-α, interferons), NO.
b) plasma mediators: complement system, clotting and fibrinolytic system, kallikrien-kinin system

The best-known forms of acute inflammation are exudative inflammation, serous inflammation, fibrinous inflammation, purulent inflammation, hemorrhagic inflammation, necrotizing and ulcerating inflammation, gangrenous inflammation and acute lymphocytic inflammation.

By contrast, a chronic inflammation typically has a long progression (weeks, months or years) often with an insidious onset and developing symptoms, especially a persistence of the damage.

An inflammatory disorder which is preferably to be treated with the aid of the preparation of the invention is paradontosis. Paradontosis is an infectious disease caused in most cases by the bacteria Porphyramonas gingivalis, Bacteroides forsythus and Actinobacillus actinomycetemcomitans. The presence of the bacteria is a necessary but not sufficient condition for the occurrence of the disease. The continuous release of harmful substances, especially lipopolysaccharides, by the bacteria activates the host's immune system and induces release of inflammatory mediators and MMPs (Matrix-Metallo-Proteases) by the monocytes. Proinflammatory cytokines such as IL-1β and TNF-α in turn activate the fibroblasts in the surrounding tissue, which themselves increase the release of MMPs. Activated macrophages and fibroblasts additionally reduce the expression of TIMPs. The consequence is an increase in the net activity of MMPs and destruction of the surrounding tissue.

In the initial stage of paradontosis, the MMP-mediated dissolution of small amounts of the connecting epithelial tissue between gum and the surface of the root of the tooth results in a pocket which creates an access for the bacteria to the lower-lying layers and thus permits the disease to progress. Decreasing the destruction of the extracellular matrix is therefore a promising approach to the treatment and prophylaxis of periodontosis.

The nucleic acids supplied to the epithelial integument with the aid of the preparation of the invention ensure suppression of the excessive immune response in the epithelial integument and thus ensure a controlled balance between synthesis and breakdown of collagen.

The preparation of the invention is additionally suitable for the prophylaxis and treatment of various other disorders or unwanted conditions, especially inflammation-related aging processes, psoriasis, atopic eczema, “dry skin”, alopecia greata, vitiligo, bullous disorders, rejection reactions (graft-versus-host reactions), UV-radiated skin inflammations, and disturbances of the function of the epidermal barrier, which are listed on page 2 of WO 98/32444, which is incorporated herein by reference.

In contrast to steroid therapy, no unwanted effects are to be expected on use of the preparation of the invention, because CpGs are naturally occurring DNA motifs.

The nucleic acids based on non-methylated CpG motifs which can be employed according to the invention can be chemically synthesized or obtained from biological sources, especially from bacteria, in a manner known to the skilled worker.

The efficacy of nucleic acids in formulations for use in particular on the skin depends on the availability of the nucleic acids in the living cells of the skin: The penetration of a macromolecule through the stratum corneum (natural barrier of the skin) into the skin is not always ensured. Nucleic acids packaged in liposomes are, however, able to penetrate the stratum corneum of skin models. Preparations preferred according to the invention are therefore those which comprise the nucleic acids based on non-methylated CpG motifs which can be employed according to the invention packaged in liposomes.

Suitable liposomes are prepared particularly preferably as described in DE-A 197 40 092, which is incorporated herein by reference.

The present invention further relates to the use of nucleic acids based on non-methylated CpG motifs for the prophylaxis and/or treatment of epithelial integument, in particular for the prophylaxis and/or treatment of epithelial integument with inflammatory changes.

The present invention further relates to a process for producing a cosmetic or pharmaceutical preparation, in particular for the prophylaxis and/or treatment of epithelial integument with inflammatory changes, characterized in that nucleic acids based on non-methylated CpG motifs, as described for the preparations of the invention, are mixed with cosmetically and pharmacologically suitable and acceptable carriers.

The present invention further relates to fabric softeners, handwashing compositions, body- and hair-care compositions, hair-coloring compositions or manual dishwashing compositions which include nucleic acids based on non-methylated CpG motifs, as described for the preparations of the invention.

The nucleic acids based on non-methylated CpG motifs are, for the purposes of the present invention, preferably introduced or incorporated as component in a cosmetic or pharmaceutical preparation or in fabric softeners, handwashing compositions, manual dishwashing compositions, or body-care compositions.

Depending on the nature of the formulation, the pharmaceutical preparations of the invention may comprise at least one further excipient or additive such as, for example, oils, protective colloids, emollients, antioxidants and/or emulsifiers. In the case of a dispersion, especially in the case of a suspension or emulsion, it is advantageous additionally to use a physiologically tolerated oil such as, for example, sesame oil, corn germ oil, cottonseed oil, soybean oil or peanut oil, esters of medium chain-length vegetable fatty acids or fish oils such as, for example, mackerel, sprat or salmon oil.

To increase the stability of the active ingredient against oxidative breakdown, it is advantageous to add stabilizers such as α-tocopherol, t-butylhydroxytoluene, t-butyl-hydroxyanisole, ascorbic acid or ethoxyquins.

The dosage and duration of use of the nucleic acids based on non-methylated CpG motifs which can be employed according to the invention can be adapted and varied in a suitable manner by the skilled worker.

The fabric softeners, handwashing compositions and manual dishwashing compositions, and the cosmetic preparations, body- and hair-care compositions, and hair-coloring compositions such as, for example, hair shampoos, hair lotions, foam baths, shower baths, creams, gels, lotions, alcoholic and aqueous/alcoholic solutions, emulsions, wax/fat compositions, stick products, dusting powders or ointments of the invention can—depending on the nature of the formulation—comprise as excipients and additives, mild surfactants, oily substances, emulsifiers, superfatting agents, pearlescent waxes, bodying agents, thickeners, polymers, silicone compounds, fats, waxes, stabilizers, biogenic active ingredients, deodorants, antiperspirants, antidandruff agents, film formers, swelling agents, UV light protection factors, antioxidants, hydrotropic agents, preservatives, insect repellants, self-tanning agents, solubilizers, perfume oils, colorants and the like.

Typical examples of suitable mild surfactants, i.e. those particularly well tolerated by skin, are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides, fatty acid glucamides, alkylamidobetaines and/or protein-fatty acid condensates, the latter preferably based on wheat proteins.

Examples of suitable oily substances are guerbet alcohols based on fatty alcohols having 6 to 18, preferably 8 to 10, carbon atoms, esters of linear C₆-C₂₂ fatty acids with linear C₆-C₂₂ fatty alcohols, esters of branched C₆-C₁₃ carboxylic acids with linear C₆-C₂₂ fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl erucate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate.

Also suitable are esters of linear C₆-C₂₂ fatty acids with branched alcohols especially 2-ethylhexanol, esters of hydroxy carboxylic acids with linear or branched C₆-C₂₂ fatty alcohols, especially dioctyl malate, esters of linear and/or branched fatty acids with polyhydric alcohols (such as, for example, propylene glycol, dimerdiol or trimertriol) and/or guerbet alcohols, triglycerides based on C₆-C₁₀ fatty acids, liquid mono/di/triglyceride mixtures based on C₆-C₁₈ fatty acids, esters of C₆-C₂₂ fatty alcohols and/or guerbet alcohols with aromatic carboxylic acids, especially benzoic acid, esters of C₂-C₁₂ dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂ fatty alcohol carbonates, guerbet carbonates, esters of benzoic acid with linear and/or branched C₆-C₂₂ alcohols (e.g. Finsolv® TN), linear or branched, symmetric or asymmetric dialkyl ethers having 6 to 22 carbon atoms per alkyl group, products of the ring opening of epoxidized fatty acid esters with polyols, silicone oils and/or aliphatic or naphthenic hydrocarbons, such as, for example, squalane, squalene or dialkylcyclohexanes.

Examples of suitable emulsifiers are nonionic surfactants from at least one of the following groups:
(1) adducts of 2 to 30 mol of ethylene oxide and/or 0 to 5 mol of propylene oxide with linear fatty alcohols having 8 to 22 C atoms, with fatty acids having 12 to 22 C atoms, with alkylphenols having 8 to 15 C atoms in the alkyl group and alkylamines having 8 to 22 carbon atoms in the alkyl radical;
(2) C_{12/18} fatty acid monoesters and diesters of adducts of 1 to 30 mol of ethylene oxide with glycerol;
(3) glycerol monoesters and diesters and sorbitan monoesters and diesters of saturated and unsaturated fatty acids having 6 to 22 carbon atoms and the ethylene oxide adducts thereof;
(4) alkyl and/or alkenyl monoglycosides and oligoglycosides having 8 to 22 carbon atoms in the alk(en)yl radical and the ethoxylated analogs thereof;
(5) adducts of 15 to 60 mol of ethylene oxide with castor oil and/or hardened castor oil;
(6) polyol esters and especially polyglycerol esters;
(7) adducts of 2 to 15 mol of ethylene oxide with castor oil and/or hardened castor oil;
(8) partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose);
(9) mono-, di- and trialkyl phosphates, and mono-, di- and/or tri-PEG-alkyl phosphates and the salts thereof;
(10) wool wax alcohols;
(11) polysiloxane-polyalkyl polyether copolymers and corresponding derivatives;
(12) mixed esters of pentaerythritol, fatty acids, citric acid and fatty alcohol as disclosed in DE 1165574 and/or mixed esters of fatty acids having 6 to 22 carbon atoms, methylglucose and polyols, preferably glycerol or polyglycerol,
(13) polyalkylene glycols and
(14) glycerol carbonate.

The adducts of ethylene oxide and/or of propylene oxide with fatty alcohols, fatty acids, alkylphenols, glycerol monoesters and diesters and sorbitan monoesters and diesters of fatty acids or with castor oil are known products which are commercially available.

They are mixtures of homologs whose average degree of alkoxylation corresponds to the ratio of the amounts of ethylene oxide and/or propylene oxide and substrate with which the addition reaction is carried out. C_{12/18} fatty acid monoesters and diesters of adducts of ethylene oxide with glycerol are disclosed in DE 2024051 as refatting agents for cosmetic preparations.

Alkyl and/or alkenyl monoglycosides and oligoglycosides, their preparation and their use are known in the art. They are prepared in particular by reacting glucose or oligosaccharides with primary alcohols having 8 to 18 C atoms. With regard to the glycoside residue, both monoglycosides in which a cyclic sugar residue is glycosidically linked to the fatty alcohol, and oligomeric glycosides having a degree of oligomerization of preferably up to about 8 are suitable. The degree of oligomerization is in this connection a statistical average which is based on a homolog distribution usual for such technical products.

Typical examples of suitable polyglycerol esters are polyglyceryl-2 dipolyhydroxystearate (Dehymuls® PGPH), polyglyceryl-3-diisostearate (Lameform® TGI), polyglyceryl-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (polyglycerol caprate T2010/90), polyglyceryl-3 cetyl ether (Chimexane® NL), polyglyceryl-3 distearate (Cremophor® GS 32) and polyglyceryl polyricinoleate (Admul® WOL 1403), polyglyceryl dimerate isostearate and the mixtures thereof.

Zwitterionic surfactants can also be used as emulsifiers. Zwitterionic surfactants refer to those surface-active compounds which have in the molecule at least one quaternary ammonium group and at least one carboxylate and one sulfonate group. Particularly suitable zwitterionic surfactants are the so-called betaines such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 C atoms in the alkyl or acyl group, and cocoacylaminoethylhydroxyethylcarboxymethyl glycinate. The fatty amide derivative known under the CTFA name Cocamidopropyl Betaine is particularly preferred. Ampholytic surfactants are likewise suitable emulsifiers. Ampholytic surfactants mean those surface-active compounds which, apart from a C_{8/18} alkyl or acyl group, comprise in the molecule at least one free amino group and at least one —COOH or —SO₃H group and are able to form inner salts. Examples of suitable ampholytic surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each having about 8 to 18 C atoms in the alkyl group. Particularly preferred ampholytic surfactants are N-cocoalkylaminopropionate, cocoacylaminoethylaminopropionate and C_{12/18} acylsarcosine. Besides ampholytic emulsifiers, quaternary ones are also suitable, with particular preference for those of the esterquat type, preferably methyl-quaternized difatty acid triethanolamine ester salts.

Superfatting agents which can be used are substances such as, for example, lanolin and lecithin, and polyethoxylated or acylated lanolin and lecithin derivatives, polyol fatty acid esters, monoglycerides and fatty acid alkanolamides, the latter simultaneously serving as foam stabilizers.

Examples of suitable pearlescent waxes are: alkylene glycol esters, specifically ethylene glycol distearate; fatty acid alkanolamides, specifically coco fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances such as, for example, fatty alcohols, fatty ketones, fatty aldehydes, fatty ethers and fatty carbonates, which have in total at least 24 carbon atoms, specifically laurone and distearyl ether; fatty acids such as stearic acid, hydroxystearic acid or behenic acid, products of the ring opening of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Bodying agents primarily suitable are fatty alcohols or hydroxy fatty alcohols having 12 to 22 and preferably 16 to 18 carbon atoms and, in addition, partial glycerides, fatty acids or hydroxy fatty acids. A combination of these substances with alkyl oligoglucosides and/or fatty acid N-methylglucamides of the same chain length and/or polyglyceryl poly-12-hydroxystearates is preferred.

Examples of suitable thickeners are Aerosil types (hydrophilic silicas), polysaccharides, especially xanthan gum, guar-guar, agar-agar, alginates and Tyloses, carboxymethylcellulose and hydroxyethylcellulose, also high molecular weight polyethylene glycol monoesters and diesters of fatty acids, polyacrylates (e.g. Carpols® from Goodrich or Synthalens® from Sigma), polyacrylamides, polyvinyl alcohol and polyvinylpyrrolidone, surfactants such as, for example, ethoxylated fatty acid glycerides, esters of fatty acids with polyols such as, for example, pentaerythritol or trimethylolpropane, fatty alcohol ethoxylates with restricted homolog distribution or alkyl oligoglucosides, and electrolytes such as sodium chloride and ammonium chloride.

Examples of suitable cationic polymers are cationic cellulose derivatives such as, for example, a quaternized hydroxyethylcellulose which is obtainable under the name Polymer JR 400® from Amerchol, cationic starch, copolymers of diallylammonium salts and acrylamides, quaternized vinylpyrrolidone/vinylimidazole polymers such as, for example, Luviquat® (BASF), condensation products of polyglycols and amines, quaternized collagen polypeptides such as, for example, lauryldimonium hydroxypropyl hydrolyzed collagen (Lamequat®L/Grünau), quaternized wheat polypeptides, polyethyleneimine, cationic silicone polymers such as, for example, amidomethicones, copolymers of adipic acid and dimethylaminohydroxypropyldiethylenetriamine (Cartaretine®/Sandoz), copolymers of acrylic acid with dimethyldiallylammonium chloride (Merquat® 550/Chemviron), polyaminopolyamides as described for example in FR 2252840 A, and the crosslinked water-soluble polymers thereof, cationic chitin derivatives such as, for example, quaternized chitosan, optionally in microcrystalline distribution, condensation products of dihaloalkyls such as, for example, dibromobutane with bisdialkylamines such as, for example, bisdimethylamino-1,3-propane, cationic guar gum such as, for example, Jaguar® CBS, Jaguar® C-17, Jaguar® C-16 supplied by Celanese, quaternized ammonium salt polymers such as, for example, Mirapol® A-15, Mirapol® AD-1, Mirapol® AZ-1 supplied by Miranol.

Examples of suitable anionic, zwitterionic, amphoteric and nonionic polymers are vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, uncrosslinked and polyol-crosslinked polyacrylic acids, acrylamidopropyltrimethylammonium chloride/acrylate copolymers, octylacrylamide/methyl methacrylate/tert-butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl methacrylate/vinylcaprolactam terpolymers, and optionally derivatized cellulose ethers and silicones.

Examples of suitable silicone compounds are dimethylpolysiloxanes, methylphenylpolysilioxanes, cyclic silicones, and amino-, fatty acid-, alcohol-, polyether-, epoxy-, fluorine-, glycoside- and/or alkyl-modified silicone compounds which may be either liquid or resinous at room temperature. Also suitable are simethicones, which comprise mixtures of dimethicones with an average chain length of from 200 to 300 dimethylsiloxane units and hydrogenated silicates. A detailed review of suitable volatile silicones by Todd et al. is also to be found in Cosm. Toil. 91, 27 (1976).

Typical examples of fats are glycerides, and suitable waxes are, inter alia, natural waxes such as, for example, candelilla wax, carnauba wax, Japan wax, esparto grass wax, cork wax, guaruma wax, rice seed oil wax, sugar cane wax, ouricury wax, montan wax, beeswax, shellac wax, spermaceti, lanolin (wool wax), preen gland fat, ceresin, ozokerite (earth wax), petrolatum, paraffin waxes, microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes.

Stabilizers which can be employed are metal salts of fatty acids, such as, for example, magnesium, aluminum and/or zinc stearate and ricinoleate.

Biogenic active ingredients mean, for example, tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, plant extracts and vitamin complexes.

Cosmetic deodorants counteract body odors, or mask or eliminate them. Body odors arise through the action of skin bacteria on apocrine sweat, forming breakdown products with an unpleasant odor. Accordingly, deodorants comprise active ingredients which act as antimicrobial agents, enzyme inhibitors, odor absorbers or odor masking agents.

Antimicrobial agents suitable for addition where appropriate to the cosmetics of the invention are in principle all substances active against Gram-positive bacteria, such as, for example, 4-hydroxybenzoic acid and its salts and esters, N-(4-chlorophenyl)-N′-(3,4-dichlorophenyl)urea, 2,4,4′-trichloro-2′-hydroxydiphenyl ether (triclosan), 4-chloro-3,5-dimethylphenol, 2,2′-methylenebis(6-bromo-4-chlorophenol), 3-methyl-4-(1-methylethyl)phenol, 2-benzyl-4-chlorophenol, 3-(4-chlorophenoxy)-1,2-propanediol, 3-iodo-2-propynyl butylcarbamate, chlorhexidine, 3,4,4′-trichlorocarbanilide (TTC), antibacterial fragrances, thymol, thyme oil, eugenol, clove oil, menthol, mint oil, farnesol, phenoxyethanol, glyceryl monolaurate (GML), diglyceryl monocaprate (DMC), N-salicylamides such as, for example, N-n-octylsalicylamide or N-n-decylsalicylamide.

Enzyme inhibitors can also be added to the cosmetics of the invention. Examples of suitable enzyme inhibitors are esterase inhibitors. These are preferably trialkyl citrates such as trimethyl citrate, tripropyl citrate, triisopropyl citrate, tributyl citrate and, in particular, triethyl citrate (Hydagen® CAT, Henkel KGaA, Düsseldorf/FRG). The substances inhibit the enzymic activity and thus reduce odor formation. Further substances which are suitable as esterase inhibitors are sterol sulfates or phosphates such as, for example, lanosterol, cholesterol, campesterol, stigmasterol and stitosterol sulfate or phosphate, dicarboxylic acids and esters thereof, such as, for example, glutaric acid, glutaric acid monoethyl ester, glutaric acid diethyl ester, adipic acid, adipic acid monoethyl ester, adipic acid diethyl ester, malonic acid and malonic acid diethyl ester, hydroxy carboxylic acids and esters thereof, for example, citric acid, malic acid, tartaric acid or tartaric acid diethyl ester, and zinc glycinate.

Substances suitable as odor absorbers are those able to absorb and substantially immobilize odoriferous compounds. They lower the partial pressure of the individual components and thus also reduce their rate of spread. It is important in this connection that perfumes remain unimpaired. Odor absorbers have no antibacterial activity. They comprise for example as main constituent a complex zinc salt of ricinoleic acid or specific, substantially odor-neutral aromatic substances which are known to the skilled worker as “fixatives”, such as, for example, extracts of labdanum or styrax or certain abietic acid derivatives. Fragrances or perfume oils act as odor-masking agents and, in addition to their function as odor-masking agents, they confer their respective scent note on the deodorants. Examples of perfume oils which may be mentioned are mixtures of natural and synthetic fragrances. Natural fragrances are extracts from flowers, stems and leaves, fruits, fruit peels, roots, woods, herbs and grasses, needles and branches, and resins and balsams. Also suitable are animal raw materials such as, for example, civet and castoreum. Typical synthetic fragrance compounds are products of the ester, ether, aldehyde, ketone, alcohol and hydrocarbon type. Examples of fragrance compounds of the ester type are benzyl acetate, p-tert-butylcyclohexyl acetate, linalyl acetate, phenylethyl acetate, linalyl benzoate, benzyl formate, allylcyclohexyl propionate, styrallyl propionate and benzyl salicylate. The ethers include, for example, benzyl ethyl ether, the aldehydes for example the linear alkanals having 8 to 18 carbon atoms, citral, citronellal, citronellyloxyacetaldehyde, cyclamen aldehyde, hydroxycitronellal, lilial and bourgeonal, the ketones for example the ionones and methyl cedryl ketone, the alcohols anethol, citronellol, eugenol, isoeugenol, geraniol, linalool, phenylethyl alcohol and terpineol, the hydrocarbons include mainly the terpenes and balsams. However, preference is given to the use of mixtures of different fragrances which together generate an agreeable scent note. Essential oils of relatively low volatility, which are mostly used as aroma components, are also suitable as perfume oils, e.g. sage oil, camomile oil, clove oil, melissa oil, mint oil, cinnamon leaf oil, lime flower oil, juniper oil, vetiver oil, olibanum oil, galbanum oil, labdanum oil and lavandin oil. Preference is given to the use of bergamot oil, dihydromyrcenol, lilial, Lyral, citronellol, phenylethyl alcohol, α-hexyl-cinnamaldehyde, geraniol, benzylacetone, cyclamen aldehyde, linalool, Boisambrene Forte, Ambroxan, indole, hedione, sandelice, lemon oil, mandarin oil, orange oil, allylamyl glycolate, cyclovertal, lavandin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romilat, irotyl and floramat, alone or in mixtures.

Antiperspirants reduce the formation of perspiration by influencing the activity of the eccrine sweat glands and thus counteract underarm wetness and body odor. Aqueous or anhydrous formulations of antiperspirants typically comprise the following ingredients:
(a) astringent active ingredients,
(b) oil components,
(c) nonionic emulsifiers,
(d) coemulsifiers,
(e) bodying agents,
(f) excipients such as, for example, thickeners or complexing agents and/or
(g) nonaqueous solvents such as, for example, ethanol, propylene glycol and/or glycerol.

Particularly suitable astringent active ingredients of antiperspirants are salts of aluminum, zirconium or zinc. Examples of such suitable active ingredients having antihydrotic activity are aluminum chloride, aluminum chlorohydrate, aluminum dichlorohydrate, aluminum sesquichlorohydrate and the complex compounds thereof, e.g. with 1,2-propylene glycol, aluminum hydroxyallantoinate, aluminum chloride tartrate, aluminum zirconium trichlorohydrate, aluminum zirconium tetrachlorohydrate, aluminum zirconium penta-chlorohydrate and the complex compounds thereof, e.g. with amino acids such as glycine.

Antiperspirants may in addition comprise conventional oil-soluble and water-soluble auxiliaries in smaller amounts. Examples of such oil-soluble auxiliaries may be:
antiflammatory, skin-protecting or fragrant essential oils,
synthetic skin-protecting active ingredients and/or
oil-soluble perfume oils.

Examples of usual water-soluble additives are preservatives, water-soluble scents, pH regulators, e.g. buffer mixtures, water-soluble thickeners, e.g. water-soluble natural or synthetic polymers such as, for example, xanthan gum, hydroxyethylcellulose, polyvinylpyrrolidone or high molecular weight polyethylene oxides.

Antidandruff agents which can be employed are climbazole, octopirox and zinc pyrithione.

Customary film formers are, for example, chitosan, microcrystalline chitosan, quaternized chitosan, polyvinylpyrrolidone, vinylpyrrolidone/vinyl acetate copolymers, polymers of the acrylic acid series, quaternary cellulose derivatives, collagen, hyaluronic acid and salts thereof and similar compounds.

Swelling agents which can be used for aqueous phases are montmorillonites, clay minerals, Pemulen and alkyl-modified Carbopol types (Goodrich). Further suitable polymers and swelling agents can be found in the review by R. Lochhead in Cosm. Toil. 108, 95 (1993).

UV protection factors mean, for example, organic substances (light protection filters) which are liquid or crystalline at room temperature and are able to absorb ultraviolet rays and emit the absorbed energy again in the form of longer wavelength radiation, e.g. heat. UVB filters may be oil-soluble or water-soluble. Examples of oil-soluble substances which should be mentioned are:
3-benzylidenecamphor and 3-benzylidenenorcamphor and its derivatives, e.g. 3-(4-methylbenzylidene)camphor as described in EP 0693471 B 1;
4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylene);
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzalmalonate;
triazine derivatives such as, for example, 2,4,6-trianilino(p-carbo-2′-ethyl-1′-hexyloxy)-1,3,5-triazine and octyl triazone as described in EP 0818450 A1, or dioctyl butamido triazone (Uvasorb® HEB);
propane-1,3-diones such as, for example, 1-(4-tert-butylphenyl)-3-(4-methoxyphenyl)propane-1,3-dione;
ketotricyclo[5.2.1.0]decane derivatives as described in EP 0694521 B1.

Suitable water-soluble substances are:
2-phenylbenzimidazole-5-sulfonic acid and its alkali metal, alkaline earth metal, ammonium, alkylammonium, alkanolammonium and glucammonium salts;
sulfonic acid derivatives of benzophenones, preferably 2-hydroxy-4-methoxybenzophenone-5-sulfonic acid and its salts;
sulfonic acid derivatives of 3-benzylidenecamphor such as, for example, 4-(2-oxo-3-bornylidenemethyl)benzene-sulfonic acid and 2-methyl-5-(2-oxo-3-bornylidene-methyl)benzenesulfonic acid and the salts thereof.

Suitable and typical UV-A filters are, in particular, derivatives of benzoylmethane such as, for example, 1-(4′-tert-butylphenyl)-3-(4′-methoxyphenyl)propane-1,3-dione, 4-tert-butyl-4′-methoxydibenzoylmethane (Parsol 1789), 1-phenyl-3-(4′-isopropylphenyl)propane-1,3-dione, and enamine compounds as described in DE 19712033 A1 (BASF). The UV-A and UV-B filters may, of course, also be employed in mixtures. Beside the soluble substances mentioned, also suitable for this purpose are insoluble light-protection pigments, namely microdisperse metal oxides or salts. Examples of suitable metal oxides are, in particular, zinc oxide and titanium dioxide and, in addition, oxides of iron, zirconium, silicon, manganese, aluminum and cerium, and mixtures thereof. Salts which can be employed are silicates (talc), barium sulfate or zinc stearate. The oxides and salts are used in the form of the pigments for skin-care and skin-protecting emulsions and decorative cosmetics. The particles should in this case have an average diameter of less than 100 nm, preferably between 5 and 50 nm and in particular between 15 and 30 nm. They may have a spherical shape, but it is also possible to employ particles having an ellipsoidal shape or one differing in another way from the spherical form. The pigments may also be surface-treated, i.e. be in hydrophilized or hydrophobized form. Typical examples are coated titanium dioxides such as, for example, titanium dioxide T 805 (Degussa) or Eusolex® T2000 (Merck). Suitable hydrophobic coating agents are in particular silicones and specifically trialkoxyoctylsilanes or simethicones. So-called micro- or nanopigments are preferably employed in sunscreen compositions. Micronized zinc oxide is preferably used. Further suitable UV-protecting filters are to be found in the review by P. Finkel in SÖFW Journal 122, 543 (1996).

Besides the two aforementioned groups of primary photo protective substances, it is also possible to employ secondary photoprotective agents of the antioxidant type which interrupt the photochemical reaction chain which is induced when UV radiation penetrates into the skin. Typical examples thereof are amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), chlorogenic acid and derivatives thereof, lipoic acid and derivatives thereof (e.g. dihydrolipoic acid), au-rothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of gum benzoin, rutinic acid and derivatives thereof, α-glycosylrutin, ferulic acid, furfurylideneglucitol, carnosine, butylhydroxytoluene, butylhydroxyanisole, nordihydroguaiaretic acid, trihydroxybutyrophenone, uric acid and derivatives thereof, mannose and derivatives thereof, superoxide dismutase, zinc and derivatives thereof (e.g. ZnO, ZnSO₄), selenium and derivatives thereof (e.g. selenomethionine), stilbenes and derivatives thereof (e.g. stilbene oxide, trans-stilbene oxide), and the derivatives, suitable according to the invention (salts, esters, ethers, sugars, nucleotides, nucleosides, peptides and lipids), of said active ingredients.

It is additionally possible according to the invention to add compounds to suppress or reduce skin disorders induced by UV radiation, in particular activators of peroxisome proliferator-activated receptors (PPAR activators), as described in WO 02/38150, which is incorporated herein by reference.

The flow behavior can be improved by employing in addition hydrotropic agents such as, for example, ethanol, isopropyl alcohol or polyols. Polyols suitable for this purpose preferably have 2 to 15 carbon atoms and at least two hydroxyl groups. The polyols may also comprise further functional groups, in particular amino groups, or be modified with nitrogen. Typical examples are
glycerol;
alkylene glycols such as, for example, ethylene glycol, diethylene glycol, propylene glycol, butylene glycol, hexylene glycol and polyethylene glycols having an average molecular weight of from 100 to 1000 daltons;
technical oligoglycerol mixtures having a degree of self-condensation of from 1.5 to 10 such as, for example, technical diglycerol mixtures having a diglycerol content of from 40 to 50% by weight;
methylol compounds such as, in particular, trimethylolethane, trimethylolpropane, trimethylolbutane, pentaerythritol and dipentaerythritol;
lower alkyl glucosides, especially those having 1 to 8 carbon atoms in the alkyl radical, such as, for example, methyl and butyl glucoside;
sugar alcohols having 5 to 12 carbon atoms such as, for example, sorbitol or mannitol;
sugars having 5 to 12 carbon atoms such as, for example, glucose or sucrose;
aminosaccharides such as, for example, glucamine;
dialcoholamines such as diethanolamine or 2-amino-1,3-propanediol.

Examples of suitable preservatives are phenoxyethanol, formaldehyde solution, parabens, pentanediol or sorbic acid, and the further classes of substances listed in Annex 6, part A and B of the cosmetic regulations. Suitable insect repellants are N,N-diethyl-m-toluamide, 1,2-pentanediol or ethyl butylacetylaminopropionate, and dihydroxyacetone is suitable as self-tanning agent.

Perfume oils which may be mentioned are mixtures of natural and synthetic fragrances. Natural fragrances are extracts of flowers (lily, lavender, rose, jasmine, neroli, ylang ylang), stems and leaves (geranium, patchouli, petitgrain), fruits (aniseed, coriander, caraway, juniper), fruit peels (bergamot, lemon, orange), roots (mace, angelica, celeriac, cardamon, costus, iris, calmus), woods (pinewood, sandalwood, guaiac wood, cedar wood, rosewood), herbs and grasses (tarragon, lemongrass, sage, thyme), needles and branches (spruce, fir, pine, dwarf pine), resins and balsams (galbanum, elemi, benzoin, myrrh, olibanum, opoponax). Also suitable are animal raw materials such as, for example, civet and castoreum. Typical synthetic fragrance compounds are products of the ester, ether, aldehyde, ketone, alcohol and hydrocarbon type. Examples of fragrance compounds of the ester type are benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. The ethers include for example benzyl ether, the aldehydes for example the linear alkanals having 8 to 18 carbon atoms, citral, citronellal, citronellyloxyacetaldehyde, cyclamen aldehyde, hydroxycitronellal, lilial and bourgeonal, the ketones for example the ionones, α-isomethylionone and methyl cedryl ketone, the alcohols anethol, citronellol, eugenol, isoeugenol, geraniol, linalool, phenylethyl alcohol and terpineol, and the hydrocarbons include mainly the terpenes and balsams. However, preference is given to the use of mixtures of different fragrances which together generate an agreeable scent note. Essential oils of relatively low volatility, which are mostly used as aroma components, are also suitable as perfume oils, e.g. sage oil, camomile oil, clove oil, melissa oil, mint oil, cinnamon leaf oil, lime flower oil, juniper oil, vetiver oil, olibanum oil, galbanum oil, labdanum oil and lavandin oil. Preference is given to the use of bergamot oil, dihydromyrcenol, lilial, Lyral, citronellol, phenylethyl alcohol, α-hexylcinnamaldehyde, geraniol, benzylacetone, cyclamen aldehyde, linalool, Boisambrene Forte, Ambroxan, indole, hedione, sandelice, lemon oil, mandarin oil, orange oil, allylamyl glycolate, cyclovertal, lavandin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat, alone or in mixtures.

Colorants which can be used are the substances suitable and approved for cosmetic purposes, as compiled in the publication “Kosmetische Färbemittel” of the Farbstoffkommission der Deutschen Forschungsgemeinschaft, Verlag Chemie, Weinheim, 1984, pp. 81-106. These colorants are normally employed in concentrations of from 0.001 to 0.1% by weight based on the complete mixture.

The body-care compositions of the invention include dental care compositions and generally compositions for care of oral hygiene (oral care products).

Toothpastes comprise for example typically:
cleaning and polishing agents such as, for example, chalk, silicas, aluminum hydroxide, aluminum silicates, calcium pyrophosphate, dicalcium phosphate, insoluble sodium metaphosphate or synthetic resin powder;
humectants such as, for example, glycerol, 1,2-propylene glycol, sorbitol, xylitol and polyethylene glycols
binders and consistency regulators, e.g. natural and synthetic water-soluble polymers and water-soluble derivatives of natural products, e.g. cellulose ethers, sheet silicates, microparticulate silicas (aerogel silicas, pyrogenic silicas)
flavorings, e.g. peppermint oil, spearmint oil, eucalyptus oil, aniseed oil, fennel oil, caraway oil, menthyl acetate, cinnamaldehyde, anethole, vanillin, thymol and mixtures of these and other natural and synthetic flavorings
sweeteners such as for example, saccharin sodium, sodium cyclamate, aspartame, acesulfame K, stevioside, monellin, glycyrrhyzin, dulcin, lactose, maltose or fructose
preservatives and antimicrobial substances such as, for example, p-hydroxybenzoic esters, sodium sorbate, triclosan, hexachlorophene, phenylsalicylic esters, thymol etc.
pigments such as, for example, titanium dioxide or colored pigments to generate colored stripes,
buffer substances, e.g. primary, secondary or tertiary alkali metal phosphates, citric acid/Na citrate
wound-healing and antiinflammatory active ingredients, e.g. allantoin, urea, azulene, panthenol, acetylsalicylic acid derivatives, plant extracts, vitamins, e.g. retinol or tocopherol.

The total content of the excipients and additives can be from 1 to 50, preferably 5 to 40, % by weight—based on the composition. The cosmetics and body-care compositions can be produced by usual cold or hot processes; the phase inversion temperature method is preferably used.

The following examples describe the invention without, however, restricting it thereto.

### EXAMPLES

### Examples 1 to 9

Both the base line and the induced release of proinflammatory cytokines was measured in in vitro models with primary keratinocytes and a keratinocyte line (HaCaT).

Inflammation was induced inter alia by irradiation with 150 mJ/cm² UVB light (Waldmann 3003 K light cabin, Waldmann, Villingen-Schwenningen, Germany), by 20 ng/ml TNFα and by anisosmolar conditions (−100 mM NaCl).

The use of UVB and TNFα for in vitro stimulation of keratinocytes is well established in dermatological research (Kippenberger S, Loitsch S M, Grundmann-Kollmann M, Simon S, Dang T A, Hardt-Weinelt K, Kaufmann R., Bernd A. Activators of Peroxisome proliferator-activated receptors protect human skin from ultraviolet-B-light-induced inflammation. J Invest Dermatol; 117:1430-1436, 2001).

The efficacy of anisosmolar conditions has likewise been shown in lung epithelial cells (Loitsch S M, von Mallinckrodt G, Kippenberger S, Steinhilber D, Wagner T O, Bargon J. Reactive oxygen intermediates are involved in II-8 production induced by hyperosmotic stress in human bronchial epithelial cells. Biochem Biophys Res Commun; 276:571-578, 2000; Hashimoto S, Matsumoto K, Gon Y, Nakayama T, Takeshita I, Horie T. Hyperosmolarity-induced interleukin-8 expression in human bronchial epithelial cells through p38 mitogen-activated protein kinase. Am J Respir Crit Care Med; 159:634-640, 1999).

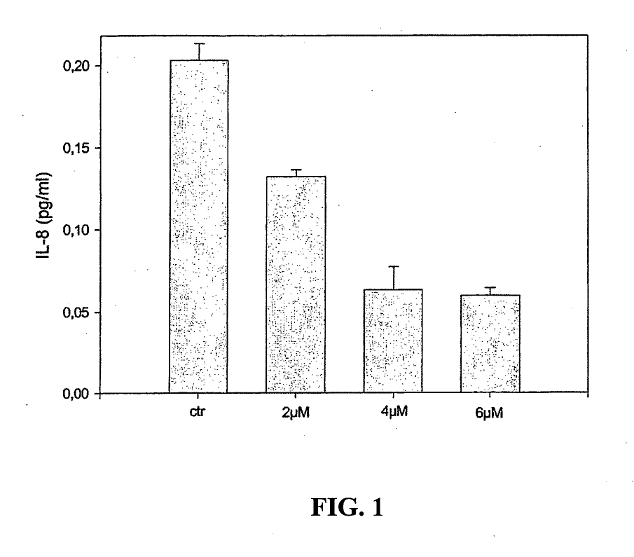

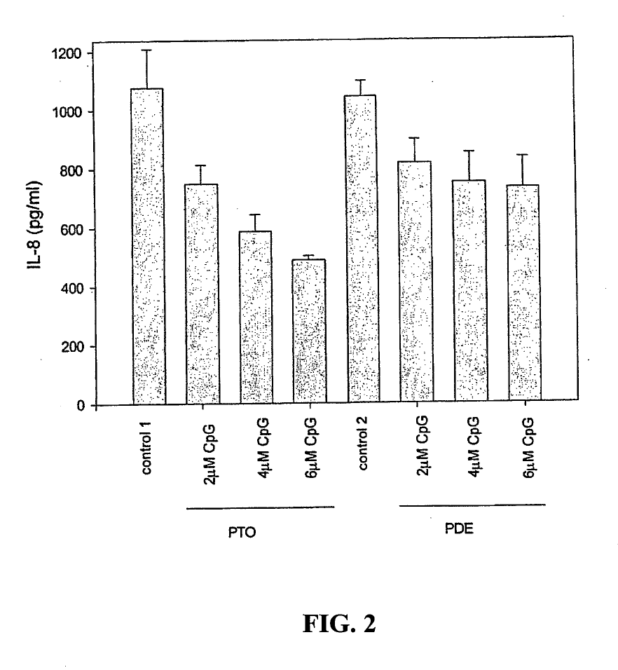

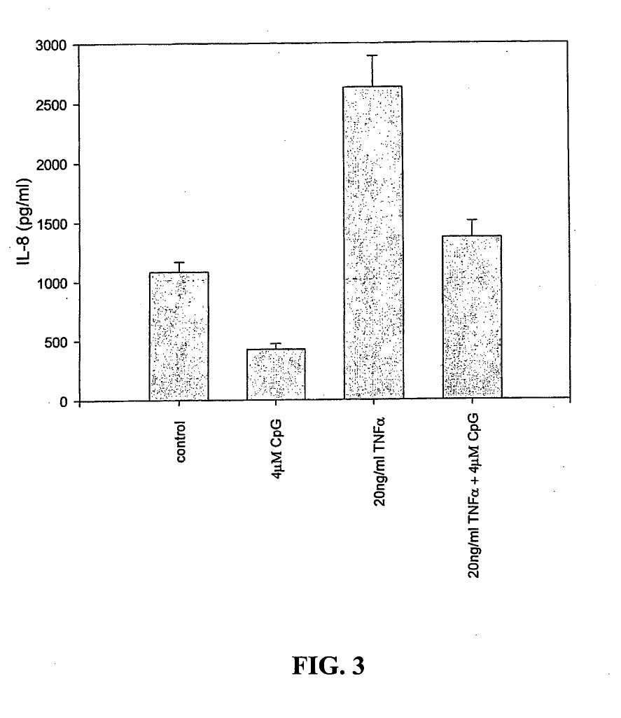

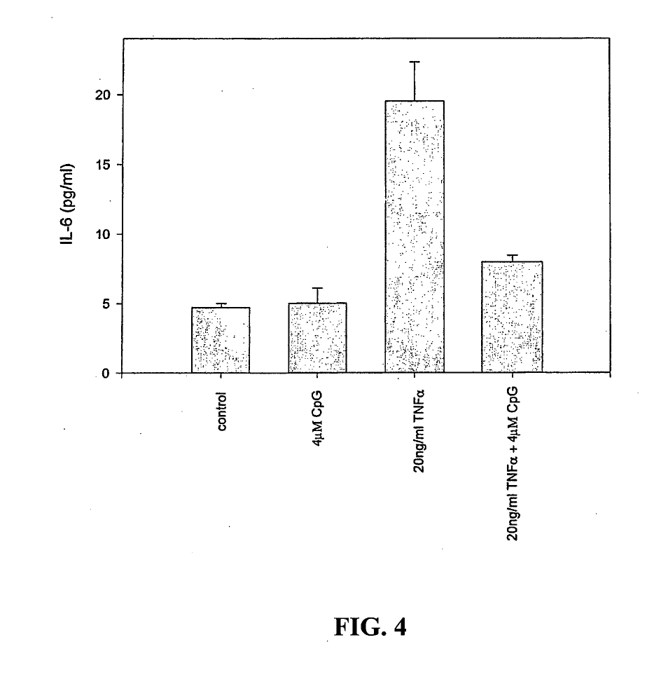

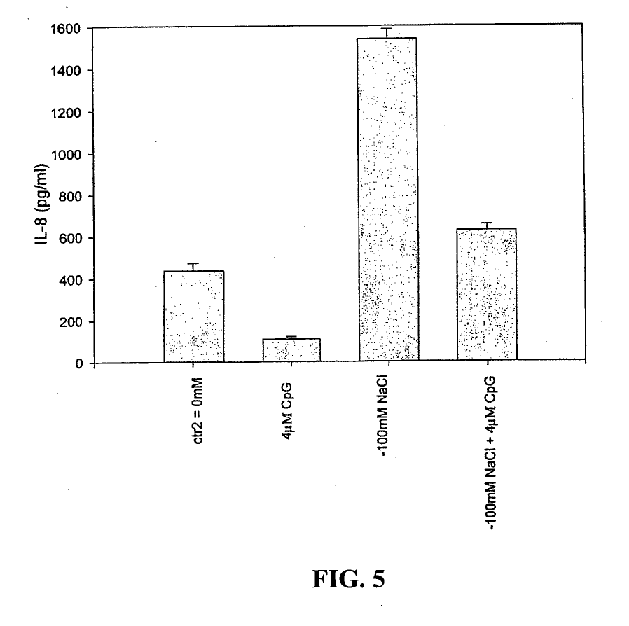

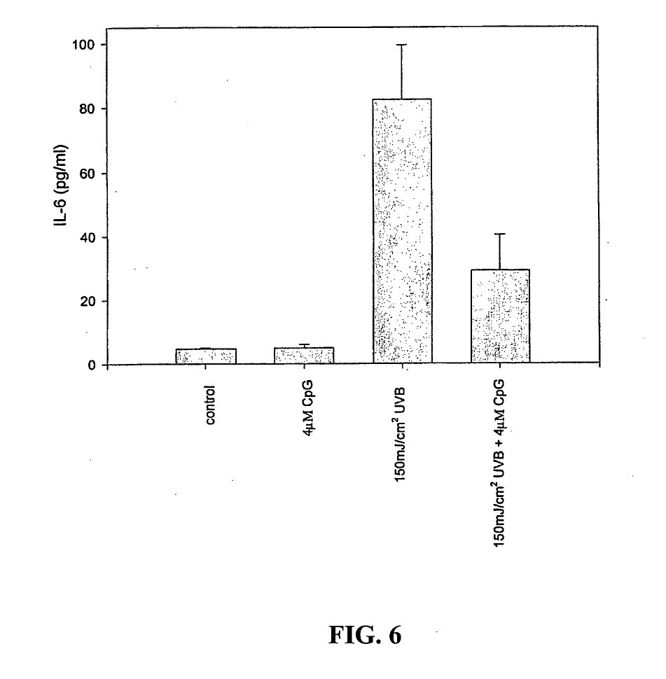

The cell species used in each case, and the design of the experiments were noted on FIGS. 1-6, which represent Examples 1-6.

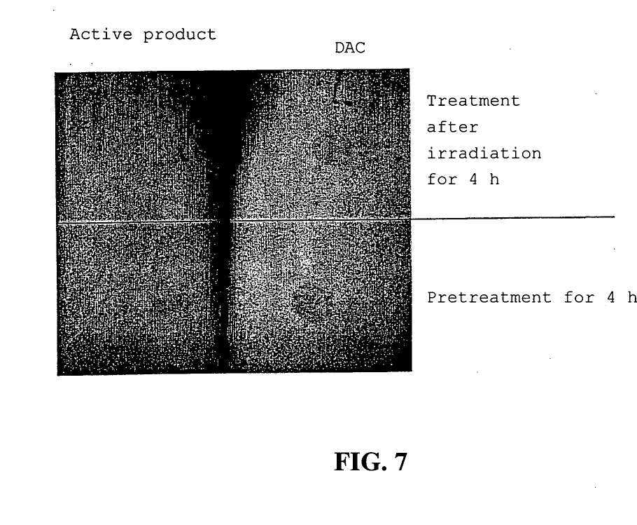

Besides the in vitro data which show immunosuppression on skin cells, this effect has also been found in self-testing in vivo (see FIG. 7). In this case, the sequence of CpG-1 (5′-TCC ATG ACG TTC CTG ACG TT-3′) (SEQ ID NO: 1) was incorporated as phosphorothioate in a concentration of 1.4% in DAC basic cream. In trial A), the active ingredient-containing cream was applied to the untreated skin for 4 h and then removed. The skin thus pretreated was then irradiated with 90 mJ/cm² UVB light (Saalmann Multitester, Saalmann, Herford, Germany). In trial B), the untreated skin was initially irradiated with 90 mJ/cm² UVB and then treated with the CpG-containing ointment for 4 h. In the controls, the skin was treated with placebo (active ingredient-free DAC base). It was found in the placebo controls that UVB light leads to clearly visible erythemas. Both the treatment before and after administration of the UVB noxae with CpG-containing cream led to distinctly less pronounced UVB erythemas. This demonstrates the surprising immunosuppressant effect of CpG on the cutaneous system.

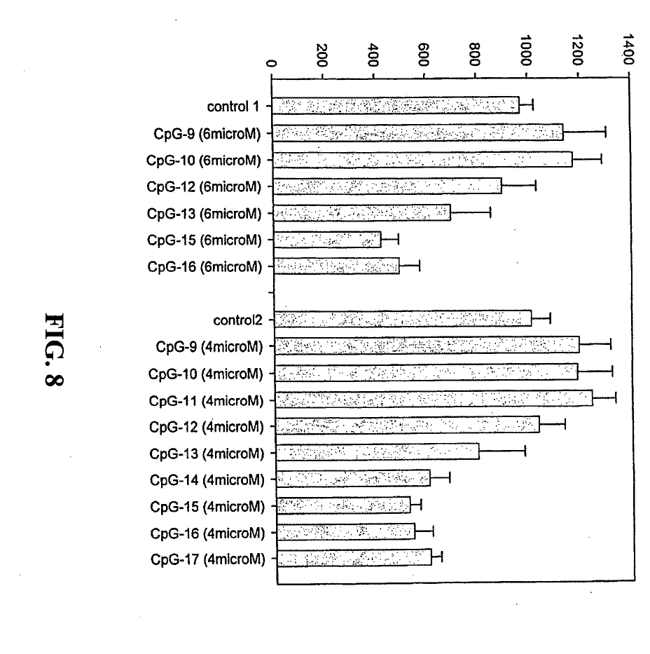

It was additionally possible to establish that the length of the oligonucleotides is important for the activity (see FIG. 8). CpG oligonucleotides derived by proximal and distal deletions of CpG-1 were tested, the sequences in detail being as follows:

| | |
|---|---|
| 1. CpG-1-PTO: | |
| 5′-TCC ATG ACG TTC CTG ACG TT-3′; | (SEQ ID NO:1) |
| 2. CpG-9-PTO: | |
| 5′-G ACG TT-3′; | (SEQ ID NO:2) |
| 3. CpG-10-PTO: | |
| 5′-TG ACG TTC-3′; | (SEQ ID NO:3) |
| 4. CpG-11-PTO: | |
| 5′-ATG ACG TTC C-3′; | (SEQ ID NO:4) |
| 5. CpG-12-PTO: | |
| 5′-C ATG ACG TTC CT-3′; | (SEQ ID NO:5) |
| 6. CpG-13-PTO: | |
| 5′-CC ATG ACG TTC CTG-3′; | (SEQ ID NO:6) |
| 7. CpG-14-PTO: | |
| 5′-TCC ATG ACG TTC CTG A-3′; | (SEQ ID NO:7) |
| 8. CpG-14A-PTO: | |
| 5′-TCC TCA ACG TTC CTG A-3′; | (SEQ ID NO:8) |
| 9. CpG-14B-PTO: | |
| 5′-TCC GCA ACG TTC CTG A-3′; | (SEQ ID NO:9) |
| 10. CpG-14C-PTO: | |
| 5′-TCC TCG ACG TCC CTG A-3′; | (SEQ ID NO:10) |
| 11. CpG-14D-PTO: | |
| 5′-TCC TCA GCG CTC CTG A-3′; | (SEQ ID NO:11) |
| 12. CpG-14E-PTO: | |
| 5′-TCC TCA ACG CTC CTG A-3′; | (SEQ ID NO:12) |
| 13. CpG-14F-PTO: | |
| 5′-TCC TCA TCG ATC CTG A-3′; | (SEQ ID NO:13) |
| 14. CpG-14G-PTO: | |
| 5′-TCC TCT TCG AAC CTG A-3′; | (SEQ ID NO:14) |
| 15. CpG-15-PTO: | |
| 5′-TCC ATG ACG TTC CTG AC-3′; | (SEQ ID NO:15) |
| 16. CpG-16-PTO: | |
| 5′-TCC ATG ACG TTC CTG ACG-3′; | (SEQ ID NO:16) |
| or | |
| 17. CpG-17-PTO: | |
| 5′-TCC ATG ACG TTC CTG ACG T-3′ | (SEQ ID NO:17) |

It was possible to show that a particularly strong immunosuppressant effect is detected when the total length is ≧14 bases.

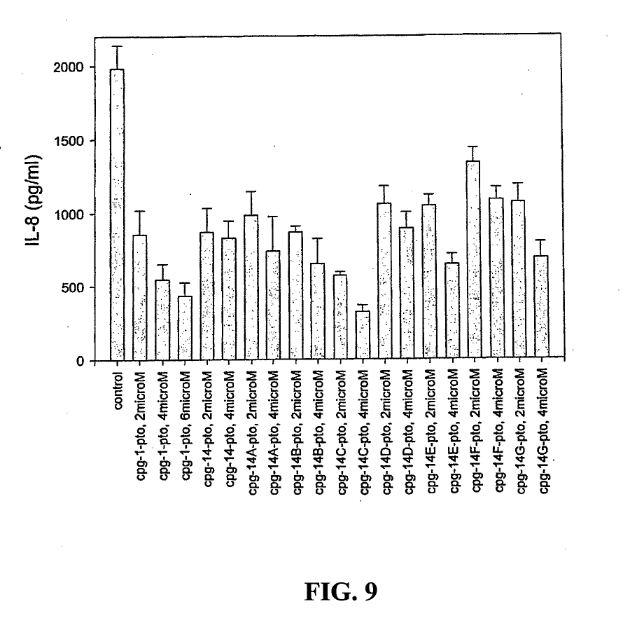

The sequences particularly preferred according to the invention include CpG-14C-PTO (see FIG. 9).

1. A method for the prophylaxis and/or treatment of epithelial integument with inflammatory changes comprising administering to a patient suffering from such a disorder a pharmaceutically acceptable amount of a nucleic acid comprising a non-methylated CpG motif.

2. The method of claim 1 wherein the inflammatory changes are exudative inflammations, serous inflammations, fibrinous inflammations, purulent inflammations, hemorrhagic inflammations, necrotizing and ulcerating inflammations, gangrenous inflammations, or acute lymphocytic inflammations.

3. The method of claim 1 wherein the inflammatory changes are caused by biological noxae or stressors, chemical noxae or stressors, or physical noxae or stressors.

4. The method of claim 3 wherein the biological noxae or stressors are pathogens, autoimmune reactions, or tumor necrosis factor; the chemical noxae or stressors are poisons or irritants; and the physical noxae or stressors are ultraviolet radiation, osmotic changes, mechanical stress, or thermal stress.

5. The method of claim 1 wherein the inflammatory changes are aging processes related to inflammation, psoriasis, atopic eczema, dry skin, alopecia greata, vitiligo, bullous disorders, rejection reactions, ultraviolet-related cutaneous inflammations, or parodontosis.

6. The method of claim 1 wherein the nucleic acid comprising a non-methylated CpG motif comprises at least one non-methylated central CG dinucleotide flanked at the 5′ end by two purine-containing nucleotides and on the 3′ end by two pyrimidine-containing nucleotides.

7. The method of claim 1 wherein the non-methylated CpG motif is

| | |
|---|---|
| 5′-TCC ATG ACG TTC CTG ACG TT-3′; | (SEQ ID NO:1) |
| 5′-G ACG TT-3′; | (SEQ ID NO:2) |
| 5′-TG ACG TTC-3′; | (SEQ ID NO:3) |
| 5′-ATG ACG TTC C-3′; | (SEQ ID NO:4) |
| 5′-C ATG ACG TTC CT-3′; | (SEQ ID NO:5) |
| 5′-CC ATG ACG TTC CTG-3′; | (SEQ ID NO:6) |
| 5′-TCC ATG ACG TTC CTG A-3′; | (SEQ ID NO:7) |
| 5′-TCC TCA ACG TTC CTG A-3′; | (SEQ ID NO:8) |
| 5′-TCC GCA ACG TTC CTG A-3′; | (SEQ ID NO:9) |
| 5′-TCC TCG ACG TCC CTG A-3′; | (SEQ ID NO:10) |
| 5′-TCC TCA GCG CTC CTG A-3′; | (SEQ ID NO:11) |
| 5′-TCC TCA ACG CTC CTG A-3′; | (SEQ ID NO:12) |
| 5′-TCC TCA TCG ATC CTG A-3′; | (SEQ ID NO:13) |
| 5′-TCC TCT TCG AAC CTG A-3′; | (SEQ ID NO:14) |
| 5′-TCC ATG ACG TTC CTG AC-3′; | (SEQ ID NO:15) |
| 5′-TCC ATG ACG TTC CTG ACG-3′; | (SEQ ID NO:16) |
| or | |
| 5′-TCC ATG ACG TTC CTG ACG T-3′. | (SEQ ID NO:17) |

8. The method of claim 7 wherein the non-methylated CpG motif is 5′-TCC TCG ACG TCC CTG A-3′ (SEQ ID NO:10).

9. The method of claim 1 wherein the nucleic acid comprising a non-methylated CpG motif is 6 to 40 nucleotides in length, 14 to 30 nucleotides in length, or 14 to 20 nucleotides in length.

10. The method of claim 1 wherein the nucleic acid comprising a non-methylated CpG motif is completely or partially chemically modified.

11. The method of claim 10 wherein the completely or partially chemically modified nucleic acid comprising a non-methylated CpG motif is chemically modified by replacement of phosphodiester bridges with methylphosphonates, phosphoramidates, phosphorothioates or hydroxylamines; replacement of riboses with hexo- or pentopyranoses or 3′-5′-carbocyclically bridged derivatives of 2′-deoxyribose; or replacement of polyester chains based on sugar-phosphate units by carboxamide chains based on amino acid derivatives.

12. The method of claim 11 wherein the amino acid derivatives are N-(2-aminoethyl)glycine units.

13. The method of claim 1 wherein the nucleic acid comprising a non-methylated CpG motif is packaged in a liposome.

14. A cosmetic, pharmaceutical, fabric softener, or manual dishwashing composition for the prophylaxis and/or treatment of epithelial integument with inflammatory changes comprising a nucleic acid comprising a non-methylated CpG motif selected from the group consisting of

| | |
|---|---|
| 5′-TCC ATG ACG TTC CTG ACG TT-3′; | (SEQ ID NO:1) |
| 5′-G ACG TT-3′; | (SEQ ID NO:2) |
| 5′-TG ACG TTC-3′; | (SEQ ID NO:3) |
| 5′-ATG ACG TTC C-3′; | (SEQ ID NO:4) |
| 5′-C ATG ACG TTC CT-3′; | (SEQ ID NO:5) |
| 5′-CC ATG ACG TTC CTG-3′; | (SEQ ID NO:6) |
| 5′-TCC ATG ACG TTC CTG A-3′; | (SEQ ID NO:7) |
| 5′-TCC TCA ACG TTC CTG A-3′; | (SEQ ID NO:8) |
| 5′-TCC GCA ACG TTC CTG A-3′; | (SEQ ID NO:9) |
| 5′-TCC TCG ACG TCC CTG A-3′; | (SEQ ID NO:10) |
| 5′-TCC TCA GCG CTC CTG A-3′; | (SEQ ID NO:11) |
| 5′-TCC TCA ACG CTC CTG A-3′; | (SEQ ID NO:12) |
| 5′-TCC TCA TCG ATC CTG A-3′; | (SEQ ID NO:13) |
| 5′-TCC TCT TCG AAC CTG A-3′; | (SEQ ID NO:14) |
| 5′-TCC ATG ACG TTC CTG AC-3′; | (SEQ ID NO:15) |
| 5′-TCC ATG ACG TTC CTG ACG-3′; | (SEQ ID NO:16) |
| and | |
| 5′-TCC ATG ACG TTC CTG ACG T-3′. | (SEQ ID NO:17) |

15. The composition of claim 14 wherein the non-methylated CpG motif is 5′-TCC TCG ACG TCC CTG A-3′ (SEQ ID NO:10).

16. The composition of claim 14 wherein the nucleic acid comprising a non-methylated CpG motif is 6 to 40 nucleotides in length, 14 to 30 nucleotides in length, or 14 to 20 nucleotides in length.

17. The composition of claim 14 wherein the nucleic acid comprising a non-methylated CpG motif is completely or partially chemically modified.

18. The composition of claim 17 wherein the completely or partially chemically modified nucleic acid comprising a non-methylated CpG motif is chemically modified by replacement of phosphodiester bridges with methylphosphonates, phosphoramidates, phosphorothioates or hydroxylamines; replacement of riboses with hexo- or pentopyranoses or 3′-5′-carbocyclically bridged derivatives of 2′-deoxyribose; or replacement of polyester chains based on sugar-phosphate units by carboxamide chains based on amino acid derivatives.

19. The composition of claim 18 wherein the amino acid derivatives are N-(2-aminoethyl)glycine units.

20. The composition of claim 14 wherein the nucleic acid comprising a non-methylated CpG motif is packaged in a liposome.

21. A process for producing a cosmetic, pharmaceutical, fabric softener, or manual dishwashing composition for the prophylaxis and/or treatment of epithelial integument with inflammatory changes comprising mixing at least one nucleic acid comprising a non-methylated CpG motif with at least one cosmetically and pharmacologically suitable and acceptable carrier.

22. The process of claim 21 wherein the non-methylated CpG motif is

| | |
|---|---|
| 5′-TCC ATG ACG TTC CTG ACG TT-3′; | (SEQ ID NO:1) |
| 5′-G ACG TT-3′; | (SEQ ID NO:2) |
| 5′-TG ACG TTC-3′; | (SEQ ID NO:3) |
| 5′-ATG ACG TTC C-3′; | (SEQ ID NO:4) |
| 5′-C ATG ACG TTC CT-3′; | (SEQ ID NO:5) |
| 5′-CC ATG ACG TTC CTG-3′; | (SEQ ID NO:6) |
| 5′-TCC ATG ACG TTC CTG A-3′; | (SEQ ID NO:7) |
| 5′-TCC TCA ACG TTC CTG A-3′; | (SEQ ID NO:8) |
| 5′-TCC GCA ACG TTC CTG A-3′; | (SEQ ID NO:9) |
| 5′-TCC TCG ACG TCC CTG A-3′; | (SEQ ID NO:10) |
| 5′-TCC TCA GCG CTC CTG A-3′; | (SEQ ID NO:11) |
| 5′-TCC TCA ACG CTC CTG A-3′; | (SEQ ID NO:12) |
| 5′-TCC TCA TCG ATC CTG A-3′; | (SEQ ID NO:13) |
| 5′-TCC TCT TCG AAC CTG A-3′; | (SEQ ID NO:14) |
| 5′-TCC ATG ACG TTC CTG AC-3′; | (SEQ ID NO:15) |
| 5′-TCC ATG ACG TTC CTG ACG-3′; | (SEQ ID NO:16) |
| or | |
| 5′-TCC ATG ACG TTC CTG ACG T-3′. | (SEQ ID NO:17) |

23. The process of claim of claim 21 wherein the at least one nucleic acid comprising a non-methylated CpG motif is completely or partially chemically modified.

24. The process of claim of claim 21 wherein the at least one nucleic acid comprising a non-methylated CpG motif is packaged in a liposome.
